(19) United States
(12) Patent Application Publication
(10) Pub. No.: US 2021/0282713 A1
(43) Pub. Date: Sep. 16, 2021

(54) SENSOR-BASED HEALTH MONITORING PLATFORM AND SENSOR EMBEDDED WEARABLE SKIN

(71) Applicant: VIRTUAL EMERGING TECHNOLOGY SOLUTIONS (V-ETS), Washington, DC (US)

(72) Inventor: JaLisa H.Y. Johnson, Arlington, VA (US)

(21) Appl. No.: 17/200,742

(22) Filed: Mar. 12, 2021

Related U.S. Application Data
(60) Provisional application No. 62/988,893, filed on Mar. 12, 2020.
(60) Provisional application No. 62/988,896, filed on Mar. 12, 2020.

(51) Int. Cl. A61B 5/00; G16H 40/67

(52) U.S. Cl. CPC A61B 5/6891; G16H 50/20; A61B 5/6801; G16H 40/67

(57) ABSTRACT

Sensor-based health data platforms and new wearable sensor devices are provided. A sensor-based health data platform includes a chair configured to receive a person for health monitoring. A first plurality of sensors are supported by the chair and configured to sense health data of a person at different locations of the person's body. A platform controller is coupled to receive sensor data from the first plurality of sensors. The platform controller is further configured to receive sensor data from a second plurality of wearable sensors worn by a person when the person is positioned in the chair for heath monitoring. A wearable skin may include the second plurality of wearable sensors embedded in the skin. A wearable sensor device has skin with multiple sensors located in different locations within the skin depending upon the skeletal structure of a human or other anatomical features that may wear the skin.

This application is a non-provisional application which claims the benefit of priority under 35 U.S.C. § 119(e) to U.S. Appl. No. 62/988,893, filed Mar. 12, 2020, and U.S. Appl. No. 62/988,896, filed Mar. 12, 2020, each of which is incorporated by reference in its entirety herein.

### BACKGROUND

### Technical Field

The technical fields relate to sensors, health data capture and monitoring, and wearable computing.

### Related Art

Health care quality can depend on the quality and quantity of data available about a patient. In the past, doctor or other health care worker would manually detect data on a patient's condition by taking a person's temperature with a thermometer or blood pressure with a blood pressure collar and record the information in a patient chart. More recently, patient information is being captured and stored in digital form using different types of sensor devices. Patient data may then be stored in electronic medical records in a database.

Applications on desktop computers and even tablet or mobile phone devices now allow authorized doctors or authorized health care workers to access and view data from a patient record. Data networking technologies now allow distributed computing and access of patient records or data from local and remote machines. Limited cloud-based services are available to provide portals for patients to access their own health data in medical records through applications on portable devices. Sensors are also being made available in wearable devices such as watches or clothing. Data sensed by these sensor can then be captured in realtime and stored for transmission to a cloud-based health data management system.

However, efforts to date have been limited. Significant gaps and technical deficiencies exist in conventional sensors and health data platforms. What is needed are new and improved sensors and health data platforms.

Significant gaps and technical deficiencies exist in conventional sensors and the integration of sensors with health data platform. Capturing data relating to human health needs to be improved.

### BRIEF SUMMARY

The present disclosure provides new sensor-based health data platforms and new wearable sensor devices. In embodiments, systems and methods are provided for sensor-based health data platforms.

A wearable sensor device has skin with multiple sensors located in different locations within the skin depending upon the skeletal structure of a human or other anatomical features that may wear the skin.

Further embodiments, features, and advantages of the invention, as well as the structure and operation of the various embodiments of the invention are described in detail below with reference to accompanying drawings.

### BRIEF DESCRIPTION OF THE FIGURES

Embodiments are described with reference to the accompanying drawings. In the drawings, like reference numbers may indicate identical or functionally similar elements. The drawing in which an element first appears is generally indicated by the left-most digit in the corresponding reference number.

FIG. 1 is an image of a health monitoring platform and a skin of wearable sensors for a person according to an embodiment of the present invention.

FIG. 2 is a wireframe diagram of a sensor-based health data platform in accordance with an embodiment of the present invention.

FIG. 3 is an image of wearable skin of sensors for a person according to a further view in an embodiment of the present invention.

FIG. 4 is a diagram of a sensor-based health data system for use in the platform of FIG. 2 and configured to be coupled to a skin of wearable sensors in accordance with an embodiment of the present invention.

### DETAILED DESCRIPTION

In one embodiment, a sensor-based health data platform is provided. The platform is a combination of hardware and software technologies that offer health monitoring and which use artificial intelligence mechanisms and sensor capabilities. The platform allows monitoring of quality of life and other health data through status information systems built into the platform (or a backbone of which the platform is a part) or wearable sensor and computing suits which can be customized to serve in different populations or applications, regardless of demographics and to serve many purposes. The platform along with wearable devices may be used in tele-home healthcare and can enhance usability, efficiency, and popularity of home-based telemedicine and allow long-term, continuous, and unobstructed monitoring of physiologic information.

In another embodiment, a wearable sensor device includes a skin having multiple sensors. The sensors are located in different locations within the skin. In one feature, the sensors are placed within the skin at locations depending upon the skeletal structure of a human that may wear the sensor device. In this way the wearable sensor device is optimized for capturing health data and the skin may be configured in a close fit or second skin for the wearer.

In one example, a wearable sensor device may also have one or more computing devices, communication devices, and a control device.

In another embodiment, a wearable sensor device may be configured to couple with an input device (such as an I/O port) into a health management system.

In a still further embodiment, a wearable sensor device may be configured to couple with a health monitoring platform. In one feature, the wearable sensor device may integrate with sensors on a health monitoring platform. This may provide an even more robust and comprehensive capture of health care data.

In one feature, the wearable senor device may be worn by first responders, soldiers, or others that may need rapid medical treatment.

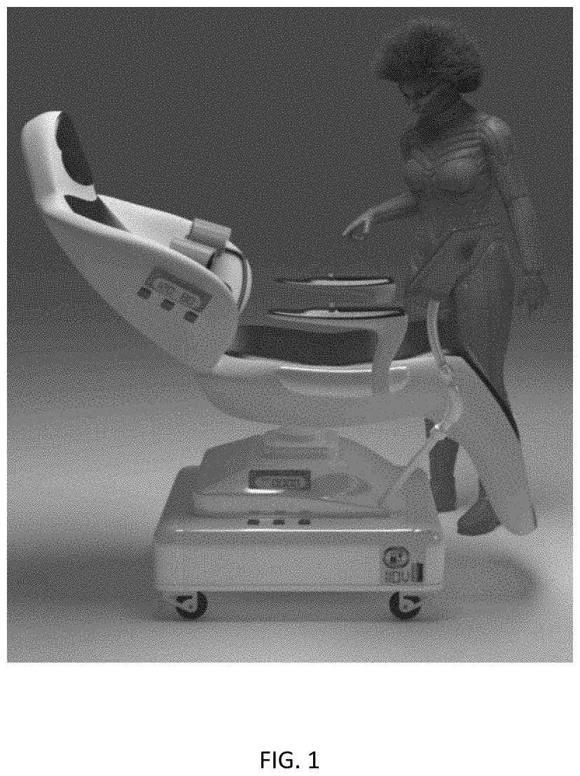

FIG. 1 is an image a sensor-based health data platform in accordance with an embodiment. The platform may be configured with one or more sensors as described further below to monitor the health of a person. As shown in the image of FIG. 1, a sensor-based health data platform may be configured to allow a person wearing a skin of wearable sensors to sit or lie on the platform. In this way, the platform may also receive data from the wearable sensors when communicatively coupled to the skin.

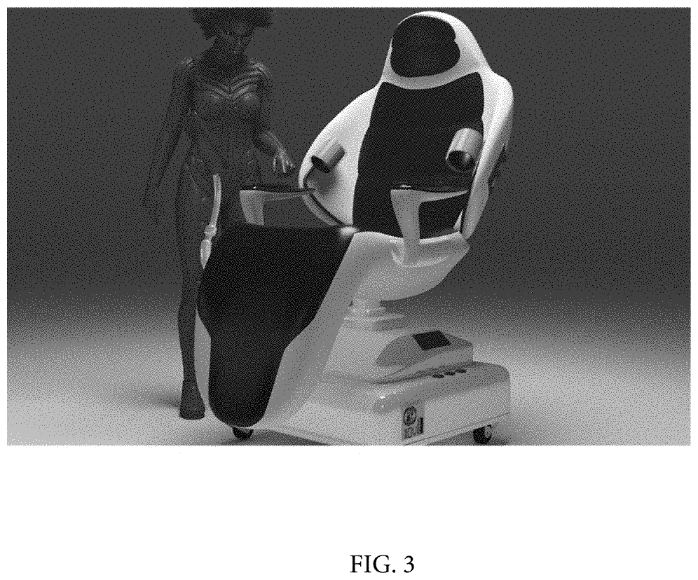

FIG. 3 is another image of wearable skin of sensors for a person according to a further view in an embodiment of the present invention. FIG. 3 shows a person having a wearable skin of sensors may be able to sit or lie in a chair or other support. A platform may receive data from the wearable skin of sensors and capture it for transmission or storage as described further below.

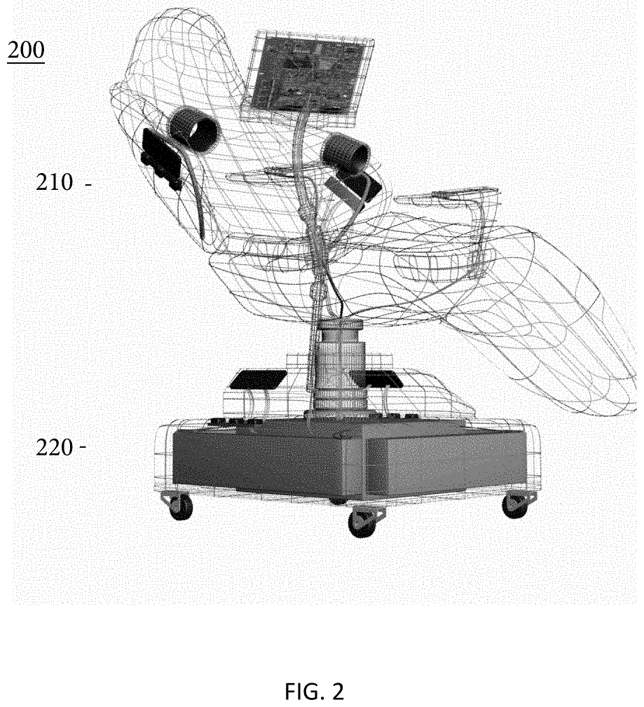

FIG. 2 is a wireframe diagram of a sensor-based health data platform 200 in accordance with an embodiment of the present invention. Platform 200 includes a chair 210 mounted on a base 220. Platform 200 may be a combination of a software and hardware technologies. Base 220 may include batteries, electronics, processors and communication links. One or more displays, user-interfaces, or other controls may be provided on base 220 and/or on chair 210 depending upon a particular application. In one example platform 200 may be mobile and base 220 may be fitted with wheels to provide mobility.

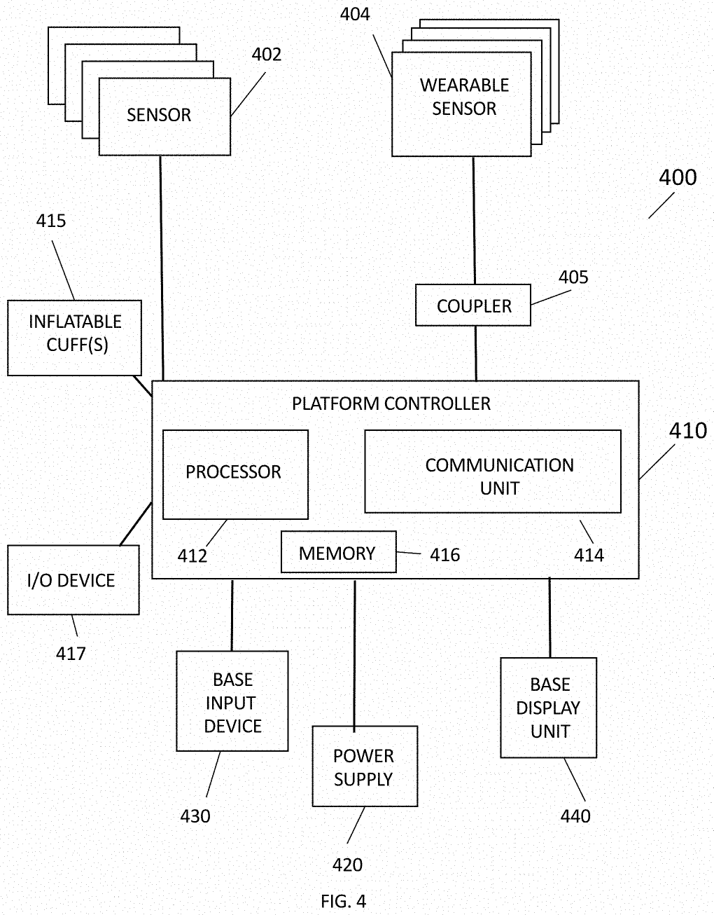

Chair 210 may allow a human or animal to sit or lie in place on platform 200. Chair 210 may be arranged in one or more different positions, including but not limited to, an upright, flat or elongated position. In one feature, chair 210 includes one or more sensors distributed on one or more locations of the chair where health conditions of a person are desired to be monitored. For example, sensors may be provided on chair 210 at locations of the chair corresponding to one or more of arms, legs, chest (such as heart or internal organs), or head. These sensors may include nanosensors or other types of sensors. Sensors may be arranged in an array or other configuration to sense data at different locations along a body in the chair. Data sensed by the sensors in the chair may be provided to the base for transmission or storage as described further below with respect to FIG. 4.

Chair 210 may also include one or more displays, user-interfaces, or other controls. In one example, a tablet device and an adjustable arm for supporting the table device may be provided on the base to support the tablet at a convenient distance for viewing and use by a user of the chair. Inflatable cuffs, restraints or other devices for supporting the capture of health data may be provided.

In a further feature, platform 200 may be configured to be part of a communications backbone with other similar platforms. The platform may also be coupled to local or remote servers, or other computing devices.

In another embodiment, platform 200 is configured to capture data from wearable devices or sensors worn by a user of the chair. In one feature, as shown in FIG. 3, a person having a wearable skin of sensors may sit or lie in the chair. The platform 200 may receive data from the wearable skin of sensors and capture it for transmission or storage.

FIG. 4 is a diagram of a sensor-based health data system 400 in platform 200 and configured to be coupled to a skin of wearable sensors 404 in accordance with an embodiment of the present invention. System 400 includes one or more sensors 402 positioned on a chair 210 of platform 200 and coupled to a platform controller 410. System 400 is also configured to be communicatively coupled to one or more wearable sensors 404. Wearable sensors 404 may be worn by person whose health is being monitored. For example wearable sensors 404 may be worn on a person's clothing or on a strap, bandage, or other fastener on a person. In one embodiment wearable sensors 404 may be embedded in a wearable skin or suit worn by a user. A coupler 405 may be provided to electrically couple wearable sensors 404 to platform controller 410. For example, coupler 405 may be a communication port that can receive sensor data from sensors 404. In another example, coupler 405 may be omitted and data from sensors 404 may be wireless sent to platform controller 410.

Platform controller 410 includes one or more processors 412, a communication unit 414, and memory 416. An input/output device 417 may also be coupled to platform controller 410. Platform controller 410 is further coupled to one or more inflatable cuffs 415. System 400 may also include a power supply 420, base input device 430, and a base display unit 440 each of which is coupled to platform controller 420.

In one embodiment, to help distribute weight toward the bottom of a platform 200, power supply 420, base input device 430, base display unit 440, and platform controller 420 may be configured as part of base 220. Inflatable cuffs 415 may be mounted on a portion of chair 210 such as the arms of chair 210 to facilitate taking of blood pressure of a person in chair 210. I/O device 417 may be any type of device allowing input from a user and display of out to a user. For example, I/O device 417 may be tablet computer, laptop, smart screen, electronic board, embedded device, or other computing device and may be removably mounted on chair 210 through a clamp, adjustable arm or stand, or other type of mount (or positioned near chair 210).

In operation, platform controller 410 may receive data from sensors 402 and wearable sensors 404. Processor 412 may control operation of system 400. Memory 416 may store sensor data received from sensors 402 and 404. Communication unit 414 may provide wired or wireless communication between platform controller 410 and a secure local or remote computing system such as a server of a cloud-based health management system. Processor 412 send sensor data from memory 416 through communication unit 414. Processor 412 may be any type of processor, microprocessor, computing device or combinations thereof. Memory 416 may be any type of computer readable memory.

I/O device 417 enables a user to further provide input such as a user selection or other controls through a user interface (such as tablet display screen) to manage health monitoring carried out by system 400 on platform 200 and enable a user to see output related to health monitoring. Base input 430 also allows a user to provide control input. For example, base input 430 may a button that may be pressed by a foot to enable power on, lock or unlocking of wheels, or other control of platform 200. Base display 440 may be a display device to display health monitoring data, system data, or other type of data for a user. Finally, power supply 420 may be any type of power supply to provide electrical power for system 400. This may include an AC or DC electrical power source or plug to a power grid, rechargeable batteries, or renewable sources such as solar panels.

While the examples above refer to chair this is not intended to be limiting. Other structures for supporting a human or animal may used. The platform may include but are not limited to, beds, tables, gurneys or other supports.

Further embodiments, features, and advantages of the invention, as well as the structure and operation of the various embodiments of the invention are described in detail below with reference to accompanying drawings.

Embodiments refer to illustrations described herein with reference to particular applications. It should be understood that the invention is not limited to the embodiments. Those skilled in the art with access to the teachings provided herein will recognize additional modifications, applications, and embodiments within the scope thereof and additional fields in which the embodiments would be of significant utility.

In the detailed description of embodiments that follows, references to “one embodiment”, “an embodiment”, “an example embodiment”, etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to effect such feature, structure, or characteristic in connection with other embodiments whether or not explicitly described.

### Further Embodiments and Example Implementations

Aspects of the embodiments may be implemented electronically using hardware, software modules, firmware, tangible computer readable or computer usable storage media having instructions stored thereon, or a combination thereof and may be implemented in one or more computer systems or other processing systems.

Embodiments may be directed to computer products comprising software stored on any computer usable medium. Such software, when executed in one or more data processing device (also called a computing device), causes a data processing device(s) to operate as described herein.

Embodiments of the present invention have been described above with the aid of functional building blocks illustrating the implementation of specified functions and relationships thereof. The boundaries of these functional building blocks have been arbitrarily defined herein for the convenience of the description. Alternate boundaries can be defined so long as the specified functions and relationships thereof are appropriately performed. The breadth and scope of the present invention should not be limited by any of the above-described exemplary embodiments.

The foregoing description of the specific embodiments will so fully reveal the general nature of the invention that others can, by applying knowledge within the skill of the art, readily modify and/or adapt for various applications such specific embodiments, without undue experimentation, without departing from the general concept of the present invention. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

What is claimed is:

1. A sensor-based health data platform comprising:
a chair configured to receive a person for health monitoring;
a first plurality of sensors supported by the chair and configured to sense health data of a person at different locations of the person's body;
a platform controller coupled to receive sensor data from the first plurality of sensors; and
wherein the platform controller is further configured to receive sensor data from a second plurality of wearable sensors worn by a person when the person is positioned in the chair for heath monitoring.

2. The platform of claim 1, further comprising a wearable skin having the second plurality of wearable sensors embedded in the skin.

3. The platform of claim 1, further comprising a coupler configured to electrically couple the second plurality of wearable sensors to the platform controller.

4. The platform of claim 1, further comprising: an input/output device coupled to the platform controller.

5. The platform of claim 1, further comprising a base coupled to support the chair.

6. The platform of claim 5, further comprising:
a base input device coupled to the platform controller; and
a base display unit coupled to the platform controller.

7. The platform of claim 1, wherein the platform controller is configured to be communicatively coupled to a local or remote health monitoring management system.

8. The platform of claim 6, wherein the base is configured to house the platform controller, the base input device, and the base display unit.

9. The platform of claim 5, the base is coupled to one or more wheels.

10. A wearable sensor device comprising a skin having multiple sensors located in different locations within the skin.

11. The device of claim 10, wherein the sensors are placed within the skin at locations depending upon the skeletal structure of a human that may wear the skin, and wherein the sensors are configured to output sensor data to a health monitoring platform.
